(19) United States
(12) Patent Application Publication
(10) Pub. No.: US 2021/0156701 A1
(43) Pub. Date: May 27, 2021

(54) INFORMATION PROCESSING APPARATUS, INFORMATION PROCESSING METHOD AND SYSTEM

(71) Applicant: TOYOTA JIDOSHA KABUSHIKI KAISHA, Toyota-shi (JP)

(72) Inventor: Koichi SUZUKI, Miyoshi-shi (JP)

(73) Assignee: TOYOTA JIDOSHA KABUSHIKI KAISHA, Toyota-shi (JP)

(21) Appl. No.: 16/950,183

(22) Filed: Nov. 17, 2020

(30) Foreign Application Priority Data
Nov. 22, 2019 (JP) 2019-211780

(51) Int. Cl. G01C 21/34; H04W 4/021; H04W 4/024; G01C 21/36; A61B 5/0205; A61B 5/00

(52) U.S. Cl. CPC G01C 21/3415; H04W 4/021; H04W 4/024; A61B 5/021; A61B 5/0205; A61B 5/4266; G01C 21/3629

(57) ABSTRACT

The present disclosure makes it possible to, when a user misses his way while moving by a vehicle, guide the user to a destination. An information processing apparatus according to the present disclosure manages a communication line for a telephone conversation between a user who is driving a vehicle and an operator who makes a telephone conversation with the user. When a current location of the vehicle is within a predetermined range from a destination of the user, the information processing apparatus executes a predetermined determination process for determining whether predetermined condition by which the user can be judged to miss his way is met. If judging that the predetermined condition is met in the predetermined determination process, the information processing apparatus notifies the operator to perform route guidance by a telephone conversation for the user.

### CROSS REFERENCE TO THE RELATED APPLICATION

This application claims the benefit of Japanese Patent Application No. 2019-211780, filed on Nov. 22, 2019, which is hereby incorporated by reference herein in its entirety.

### BACKGROUND

### Technical Field

The present disclosure relates to an information processing apparatus, an information processing method and a system.

### Description of the Related Art

In Patent document 1, a service is described in which, when a user communicates setting of a destination to an operator, using a mobile phone, information required to operate an onboard apparatus is provided via the operator. For example, a service is described in which, when the user communicates his request to the operator, the operator explains a method for setting the destination by a voice conversation, or the operator instructs an information center to transmit data for setting the destination to the onboard apparatus.

### CITATION LIST

### Patent Document

Patent document 1: Japanese Patent Laid-Open No. 2015-95736

### SUMMARY

An object of the present disclosure is to provide a technique capable of, when a user misses his way while moving by a vehicle, guiding the user to a destination.

An information processing apparatus according to the present disclosure is an information processing apparatus managing a communication line for a telephone conversation between a user who is driving a vehicle and an operator who makes a telephone conversation with the user, the information processing apparatus: executing a predetermined determination process for determining whether predetermined condition by which the user can be judged to miss his way is met when a current location of the vehicle is within a predetermined range from a destination of the user; and notifying the operator to perform route guidance by a telephone conversation for the user if judging that the predetermined condition is met in the predetermined determination process.

According to the present disclosure, it becomes possible to, when a user who is moving by a vehicle misses his way, guide the user to a destination.

### BRIEF DESCRIPTION OF THE DRAWINGS

FIG. 1 is a diagram illustrating a schematic configuration of a telephone conversation management system according to embodiments;

FIG. 2 is a block diagram schematically illustrating an example of a functional configuration of an onboard terminal in a first embodiment;

FIG. 3 is a block diagram schematically illustrating an example of a functional configuration of an operator terminal;

FIG. 4 is a block diagram schematically illustrating an example of a functional configuration of a management server in the first embodiment;

FIG. 5 is a diagram illustrating a current location of a vehicle, a destination and a determination range;

FIG. 6 is a diagram illustrating an example of a table configuration of a travel information stored in a travel information database;

FIG. 7 is a flowchart illustrating a flow of a notification process in the first embodiment;

FIG. 8 is a flowchart illustrating a flow of a notification process in a second embodiment;

FIG. 9 is a block diagram schematically illustrating an example of a functional configuration of an onboard terminal in a modification;

FIG. 10 is a block diagram schematically illustrating an example of a functional configuration of a management server in the modification; and

FIG. 11 is a diagram illustrating an example of a table configuration of a biometric information stored in a biometric information database.

### DESCRIPTION OF THE EMBODIMENTS

An information processing apparatus according to the present disclosure is an information processing apparatus that manages a communication line for a telephone conversation between a user who is driving a vehicle and an operator who makes a telephone conversation with the user. By making a telephone conversation with the operator while driving the vehicle, the user can obtain various information from the operator. Further, the operator provides the user with various information in response to a request from the user obtained by the telephone conversation.

The information processing apparatus acquires a destination of the user and a current location of the vehicle. Then, when the current location of the vehicle is within a predetermined range from the destination, a controller executes a predetermined determination process for determining whether a predetermined condition by which the user can be judged to miss his way is met or not. Here, the predetermined condition may be, for example, a condition about a traveling situation of the vehicle. Further, the predetermined condition may be a condition about biometric information about the user.

Then, when judging that the predetermined condition is met in the predetermined determination process, the controller notifies the operator to perform route guidance by a telephone conversation for the user. Thereby, it is possible to prompt the operator to perform the route guidance for the user who misses his way. As a result, it becomes possible to guide the user to the destination by the operator's telephone conversation.

The predetermined determination process is executed when the current location of the vehicle is within a predetermined range from the destination. The predetermined range refers to a geographical range that includes the destination. In this case, the controller does not execute the predetermined determination process when the current location of the vehicle is outside the predetermined range from the destination. In other words, when the current location of the vehicle is outside the predetermined range from the destination, the operator is not notified to perform route guidance by a telephone conversation for the user, by the controller. Thereby, it is possible to prevent an unnecessary telephone conversation for route guidance being performed for the user by the operator.

As described above, by the information processing apparatus according to the present disclosure, it becomes possible to, when a user who is moving by a vehicle misses his way, guide the user to a destination.

Specific embodiments of the present disclosure will be described below based on drawings. As for dimensions, materials, shapes and relative arrangement and the like of component parts described in the present embodiments, a technical scope of the present disclosure is not intended to be limited thereto unless otherwise stated.

### First Embodiment

### (Outline of System)

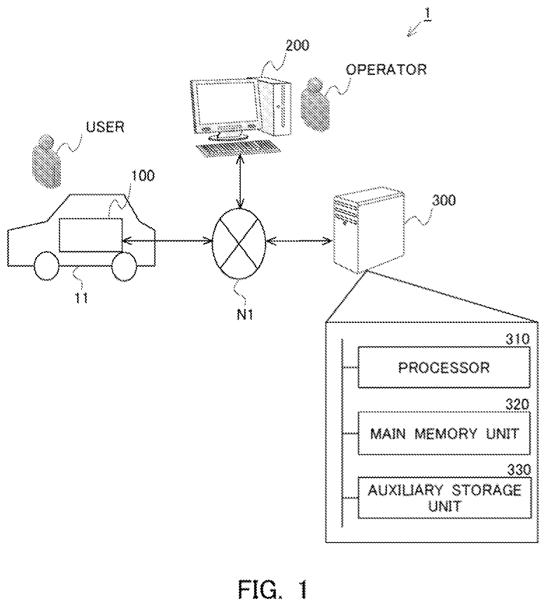

A telephone conversation management system 1 in the present embodiment will be described based on FIG. 1. FIG. 1 is a diagram illustrating a schematic configuration of the telephone conversation management system 1 according to the present embodiment. The telephone conversation management system 1 is configured, including an onboard terminal 100, an operator terminal 200 and a management server 300. The onboard terminal 100 is a terminal mounted on a vehicle 11. Here, the vehicle 11 is a vehicle that a user drives. The operator terminal 200 is a terminal that an operator uses. The management server 300 is a server that manages a communication line for a telephone conversation between the user and an operator. The onboard terminal 100, the operator terminal 200 and the management server 300 are mutually connected via a network N1. Here, for the network N1, for example, a WAN (Wide Area Network), which is a global public communication network such as the Internet, or a telephone communication network such as mobile phones and the like may be adopted.

The onboard terminal 100 is a terminal that enables a telephone conversation between the user and the operator by connecting to the operator terminal 200 via the network N1. Note that the onboard terminal 100 is not limited to a terminal that is integrally mounted on the vehicle 11. For example, a mobile information terminal that the user possesses may function as the onboard terminal 100. The operator terminal 200 is a terminal that enables a telephone conversation between the user and the operator by connecting to the onboard terminal 100 via the network N1.

The user and the operator can mutually perform a telephone conversation using the onboard terminal 100 and the operator terminal 200, respectively. The operator makes a response corresponding to content of an inquiry from the user through the telephone conversation with the user. For example, the operator can recommend a restaurant or a facility corresponding to the user's request by the telephone conversation. The operator may directly set the restaurant or the facility recommended to the user for a car navigation system (a navigation unit 103 described later) mounted on the onboard terminal 100 as the destination of the vehicle 11 via the network N1. At this time, a case may happen where the user misses his way while he is going to the user's destination (which may be hereinafter referred to simply as “destination”) by the vehicle 11. Therefore, the management server 300 performs a predetermined determination process for determining whether the user misses his way or not. Then, if the user misses his way, the management server 300 notifies the operator to perform route guidance for the user by a telephone conversation. The predetermined determination process will be described later.

The management server 300 is configured, including a general computer. The computer constituting the management server 300 is provided with a processor 310 such as a CPU and a DSP, a main memory unit 320 such as a read-only memory (ROM) and a random access memory (RAM), an auxiliary storage unit 330 such as an EPROM, a hard disk drive (HDD) and a removal medium. Here, the removable medium is, for example, a flash memory such as a USB memory and an SD card, or a disk recording medium such as a CD-ROM, a DVD disk and a Blu-ray disk.

In the management server 300, an operating system (OS), various kinds of programs, various kinds of information tables and the like are stored in the auxiliary storage unit 330. In the management server 300, by the processor 310 loading a program stored in the auxiliary storage unit 330 to the main memory unit 320 and executing the program, various kinds of functions as described later can be realized. However, a part or all of the functions in the management server 300 may be realized by a hardware circuit such as an ASIC and an FPGA. Note that the management server 300 does not necessarily have to be realized by a single physical configuration but may be configured with a plurality of mutually cooperating computers.

(System Configuration)

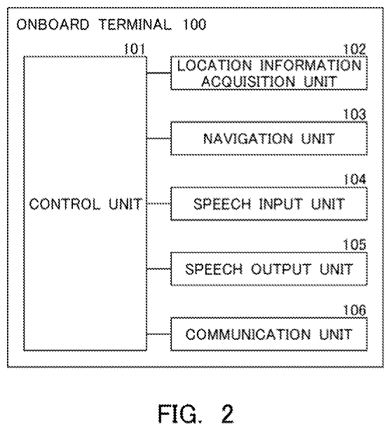

Next, a functional configuration of each of the onboard terminal 100, the operator terminal 200 and the management server 300 included in the telephone conversation management system 1 according to the present embodiment will be described based on FIGS. 2 to 4.

(Onboard Terminal)

FIG. 2 is a block diagram schematically illustrating an example of the functional configuration of the onboard terminal 100. As illustrated in FIG. 2, the onboard terminal 100 is configured, including a control unit 101, a location information acquisition unit 102, the navigation unit 103, a speech input unit 104, a speech output unit 105 and a communication unit 106. The control unit 101 has a function of performing arithmetic processing for controlling the onboard terminal 100. The control unit 101 is configured, for example, with a microcomputer.

The location information acquisition unit 102 is a device that acquires a current location of the vehicle 11. Specifically, the location information acquisition unit 102 is configured, including a GPS receiver and the like. The control unit 101 transmits current location information indicating the current location of the vehicle 11, which has been acquired by the location information acquisition unit 102, to the management server 300 via the communication unit 106.

Specifically, the navigation unit 103 is configured, including a car navigation system. The operator can set a destination of the user for the navigation unit 103 by communicating with the navigation unit 103 via the network N1 using the operator terminal 200. Note that the user himself can set the destination for the navigation unit 103. Based on the set destination, the navigation unit 103 generates a scheduled traveling route which is a traveling route of the vehicle 11 from the current location of the vehicle 11 to the destination. Furthermore, the navigation unit 103 generates route information which is information indicating the scheduled traveling route. The generated route information is displayed on a display unit that the navigation unit 103 has. Based on the route information displayed on the display unit that the navigation unit 103 has, the user can cause the vehicle 11 to travel and go to the destination. Further, the navigation unit 103 estimates scheduled arrival time of arriving at the destination in the case of the vehicle 11 traveling on the scheduled traveling route. Then, the navigation unit 103 generates scheduled time information which is information indicating the scheduled arrival time. Further, if the vehicle 11 travels on a road other than the scheduled traveling route, the navigation unit 103 regenerates a scheduled traveling route. Here, generation of the scheduled traveling route and estimation of the scheduled arrival time may be performed not by the navigation unit 103 but by a server apparatus different from the onboard terminal 100. Further, the control unit 101 transmits information indicating the destination that the operator (or the user) has set by the navigation unit 103, to the management server 300 via the communication unit 106 as destination information. Further, the control unit 101 transmits the route information and the scheduled time information that the navigation unit 103 has generated, to the management server 300 via the communication unit 106.

The speech input unit 104 is a device that converts a speech uttered by the user to speech data. Specifically, the speech input unit 104 is configured, including a microphone and the like. The control unit 101 transmits the speech data converted by the speech input unit 104 to the operator terminal 200 via the communication unit 106.

The speech output unit 105 is a device that converts the speech data to a speech. The speech output unit 105 converts the speech data that the control unit 101 has received from the operator terminal 200 or the management server 300 via the communication unit 106, to a speech. Further, the speech output unit 105 also performs output of the converted speech. Specifically, the speech output unit 105 is configured, including speakers, a headphone or earphones.

The communication unit 106 is a communication device for connecting the onboard terminal 100 to the network N1. The communication unit 106 is configured, for example, including a LAN (Local Area Network) interface board or a wireless communication circuit for wireless communication.

(Operator Terminal)

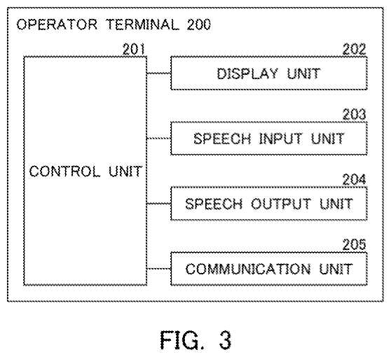

FIG. 3 is a block diagram schematically illustrating an example of the functional configuration of the operator terminal 200. As illustrated in FIG. 3, the operator terminal 200 is configured, including a control unit 201, a display unit 202, a speech input unit 203, a speech output unit 204 and a communication unit 205. The control unit 201 has a function of performing arithmetic processing for controlling the operator terminal 200. The control unit 201 is configured, for example, with a microcomputer.

The display unit 202 is configured, for example, with a liquid crystal display or an organic electroluminescent display. As described later, the operator terminal 200 receives notification information that notifies the operator to perform route guidance for the user by a telephone conversation, from the management server 300. When receiving this notification information, the control unit 201 displays the notification information on the display unit 202.

The speech input unit 203 is a device that converts a speech uttered by the operator to speech data. The control unit 201 transmits the speech data converted by the speech input unit 203 to the onboard terminal 100 via the communication unit 205. The speech output unit 204 converts speech data that the control unit 201 has received from the onboard terminal 100 via the communication unit 205, to a speech. Further, the speech output unit 204 also performs output of the converted speech.

The communication unit 205 is a communication device for connecting the operator terminal 200 to the network N1. A configuration of the communication unit 205 may be similar to the configuration of the communication unit 106 in the onboard terminal 100. The control unit 201 receives the notification information from the management server 300 via the communication unit 205 through the network N1.

By transmission and receiving of speech data obtained by converting a speech uttered by the user and speech data obtained by converting a speech uttered by the operator being performed between the onboard terminal 100 and the operator terminal 200 as described above, a telephone conversation between the user and the operator is enabled. Then, when the telephone conversation with the operator is enabled, it becomes possible for the user to communicate his request to the operator by the telephone conversation. Furthermore, the operator can select a place corresponding to the request communicated from the user and set the selected place for the navigation unit 103 of the onboard terminal 100 as a destination of the user.

(Management Server)

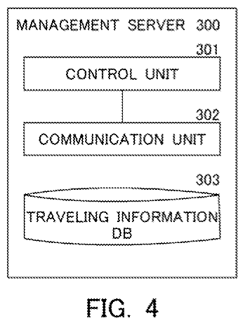

FIG. 4 is a block diagram schematically illustrating an example of the functional configuration of the management server 300. As illustrated in FIG. 4, the management server 300 is configured, including a control unit 301, a communication unit 302 and a traveling information database (a traveling information DB) 303.

The communication unit 302 is a communication device for connecting the management server 300 to the network N1. A configuration of the communication unit 302 may be similar to the configuration of the communication unit 106 in the onboard terminal 100. The control unit 301 receives the destination information, the current location information, the route information and the scheduled time information from the onboard terminal 100 via the communication unit 302. Further, the control unit 301 transmits the notification information generated by the control unit 301 to the operator terminal 200 via the communication unit 302.

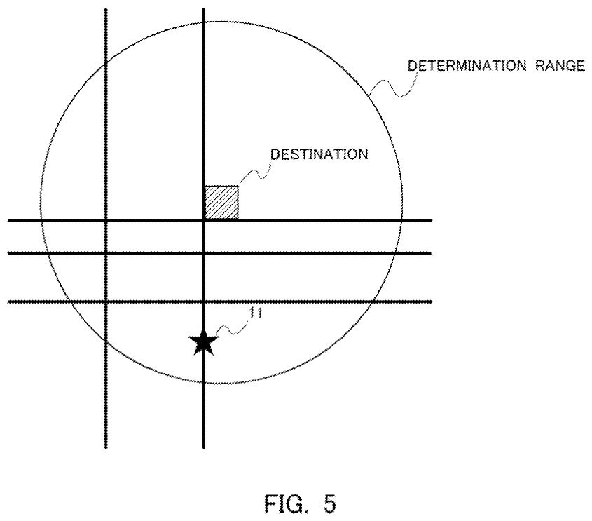

The control unit 301 has a function of performing arithmetic processing for controlling the management server 300. The control unit 301 is configured, for example, with a microcomputer. The control unit 301 sets a predetermined range (which may be hereinafter referred to as “determination range”) with the destination as a reference, based on the destination information received via the communication unit 302. Note that a reason why the control unit 301 sets the determination range will be described later. FIG. 5 is a diagram illustrating the current location of the vehicle 11, the destination and the determination range. A star illustrated in FIG. 5 indicates the current location of the vehicle 11. Lines illustrated in FIG. 5 indicate roads where the vehicle 11 can travel. As illustrated in FIG. 5, the determination range is set as a predetermined range with a destination as a reference. Note that, though the determination range illustrated in FIG. 5 has a circular shape, the determination range is not limited to a circular shape. The determination range may be a particular area that includes the destination, or a city, a town, a village or the like.

The control unit 301 continuously receives the current location information via the communication unit 302. Then, when the current location of the vehicle 11 is within the determination range, the control unit 301 generates traveling history information, which is information about transition of the current location of the vehicle 11, based on the continuously received current location information. Further, the control unit 301 receives the scheduled time information indicating scheduled arrival time estimated when the vehicle 11 enters the determination range, from the onboard terminal 100. Further, after the vehicle 11 enters the determination range, the control unit 301 continuously receives the route information from the onboard terminal 100 via the communication unit 302.

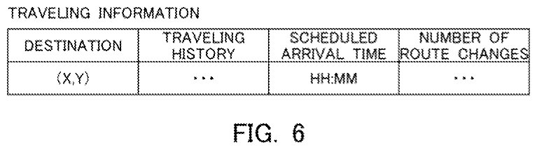

The control unit 301 stores the destination information, the traveling history information and the scheduled time information into the traveling information DB 303 as traveling information. Further, if the scheduled traveling route of the vehicle 11 included in the route information received from the onboard terminal 100 is changed by regeneration, the control unit 301 counts the number of changes and stores the number of changes into the traveling information DB 303 as traveling information. FIG. 6 is a diagram illustrating an example of a table configuration of a travel information stored in the traveling information DB 303. As illustrated in FIG. 6, the travel information table has a destination field, a traveling history field, a scheduled arrival time field and a number-of-route-changes field. In the destination field, the destination obtained from the destination information is inputted. In the traveling history field, the traveling history information is inputted. In the scheduled arrival time field, the scheduled time information is inputted. In the number-of-route-changes field, the number of times the scheduled traveling route of the vehicle 11 has been changed is inputted. In the destination field, the destination set by the operator (or the user) is inputted. In the scheduled arrival time field, scheduled arrival time estimated when the vehicle 11 enters the determination range is inputted. Therefore, the information inputted in these fields is not updated. On the other hand, a traveling history of the vehicle 11 changes as time passes. Therefore, the traveling history information inputted in the traveling history field is updated as time passes. Further, each time a scheduled traveling route is regenerated by the navigation unit 103 of the onboard terminal 100, the number of changes in the scheduled traveling route inputted in the number-of-route-changes field is also updated.

Note that, in the present embodiment, the onboard terminal 100 and the operator terminal 200 mutually transmit and receive speech data via the network N1. The onboard terminal 100 and the operator terminal 200 also transmit and receive various kinds of information to and from the management server 300 via the network N1. However, a communication network used to transmit and receive speech data between the onboard terminal 100 and the operator terminal 200 and a communication network used to transmit and receive various kinds of information between the onboard terminal 100 and the management server 300 and between the operator terminal 200 and the management server 300 may be different communication networks. For example, a WAN and a telephone communication network may be adopted as the communication network used to transmit and receive various kinds of information and the communication network used to transmit and receive speech data, respectively.

(Predetermined Determination Process)

In the management server 300, the control unit 301 determines whether a traveling situation of the vehicle 11 satisfies a predetermined traveling condition or not as the predetermined determination process for determining whether the user misses his way or not. The determination about whether the traveling situation of the vehicle 11 satisfies a predetermined traveling condition or not is performed based on the traveling information stored in the traveling information DB 303. The predetermined traveling condition is a condition by which the user can be judged to miss his way. The “predetermined traveling condition” at this time will be specifically described below.

For example, when the user misses his way, he may stop the vehicle 11 once and see a map to confirm a correct route or may operate the car navigation system. In such a case, the vehicle 11 stops at a location different from a destination for a while. At this time, the control unit 301 can grasp a time during which the vehicle 11 stops at the location different from the destination by the traveling history information included in the traveling information. Therefore, the predetermined traveling condition may be that the vehicle 11 stops at a location different from a destination for a predetermined time or more.

Further, when the user misses his way, he may turn the vehicle 11 to change a direction. At this time, the control unit 301 can determine whether the vehicle 11 has turned or not by the traveling history information included in the traveling information. Therefore, the predetermined traveling condition may be that the vehicle 11 turns.

Further, when the user misses his way, he may cause the vehicle 11 to enter a one-way road from an opposite direction. The control unit 301 can determine whether the vehicle 11 has entered the one-way road from the opposite direction or not by the traveling history information included in the traveling information. Therefore, the predetermined traveling condition may be that the vehicle 11 enters a one-way road from an opposite direction.

Further, when the user misses his way, the vehicle 11 may travel away from the destination. The control unit 301 can calculate transition of a distance between a current location of the vehicle 11 and the destination by the destination information and the traveling history information that are included in the traveling information. At this time, the distance between the current location of the vehicle 11 and the destination may be a distance of the shortest route from the current location of the vehicle 11 to the destination. Then, the control unit 301 can determine whether the vehicle 11 travels away from the destination or not based on transition of the distance between the current location of the vehicle 11 and the destination. Therefore, the predetermined traveling condition may be that the vehicle 11 travels away from a destination.

Further, when the user misses his way, the vehicle 11 may travel on a road or an intersection where the vehicle 11 has traveled once, again. The control unit 301 can determine whether the vehicle 11 travels on the road or the intersection where the vehicle 11 has traveled once, again or not based on the traveling history information included in the traveling information. Therefore, the predetermined traveling condition may be that the vehicle 11 travels on a road or an intersection where the vehicle 11 has traveled once, again.

Further, when the user misses his way, the vehicle 11 may not arrive at the destination even if the scheduled arrival time at which the vehicle 11 is to arrive the destination of the vehicle 11 has passed, the scheduled arrival time having been estimated when the vehicle 11 enters the determination range. Therefore, the predetermined traveling condition may be that the vehicle 11 has not arrived at a destination even if a predetermined time passes after a scheduled arrival time at which the vehicle 11 is to arrive at the destination, the scheduled arrival time having been estimated when the vehicle 11 enters the determination range.

Further, when the user misses his way, the vehicle 11 may travel on a route deviated from a scheduled traveling route generated when the vehicle 11 enters the determination range. Then, regeneration of the scheduled traveling route may be repeatedly performed by the navigation unit 103 in the onboard terminal 100. When regeneration of the scheduled traveling route is repeatedly performed, the number of changes in the scheduled traveling route inputted in the number-of-route-changes field of the traveling information DB 303 increases. Therefore, the predetermined traveling condition may be that route information is changed a predetermined number of times or more.

If the traveling situation of the vehicle 11 satisfies at least any one of the seven predetermined traveling conditions described above, it can be judged that the user misses his way. Therefore, in the present embodiment, when the vehicle 11 exists in the determination range, it is judged whether the traveling situation of the vehicle 11 satisfies at least any one of the seven predetermined traveling conditions described above or not. Note that the seven predetermined traveling conditions described above are examples of the traveling condition for determining whether the user misses his way or not. As the predetermined traveling condition for determining whether the user misses his way or not, other known conditions can be adopted.

(Notification to Operator)

In the present embodiment, if it is judged that the traveling situation of the vehicle 11 existing in the determination range satisfies a predetermined traveling condition in the predetermined judgment process, the management server 300 notifies the operator to perform route guidance for the user by a telephone conversation.

(Flow of Notification Process)

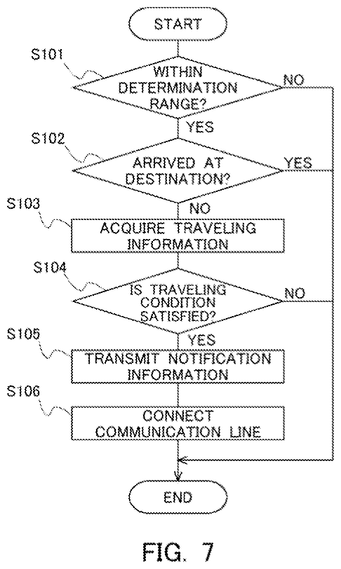

Here, a notification process executed by the control unit 301 in the management server 300 will be described based on FIG. 7. FIG. 7 is a flowchart illustrating a flow of the notification process in the present embodiment. Note that the process illustrated in FIG. 7 is cyclically executed at predetermined intervals. In the notification process, first, it is determined at S101 whether the current location of the vehicle 11 is within the determination range or not based on the current location information about the vehicle 11. If a negative judgment is made at S101, the notification process is ended once. In this case, transmission of the notification information from the management server 300 to the operator terminal 200 is not performed. Therefore, a notification prompting the operator to perform route guidance for the user by a telephone conversation is not given. On the other hand, if a positive judgment is made at S101, a process of S102 is executed.

At S102, it is judged whether the vehicle 11 has arrived at the destination or not based on the current location information about the vehicle 11. If a positive judgment is made at S102, the notification process is ended once. On the other hand, if a negative judgment is made at S102, a process of S103 is executed. At S103, the traveling information stored in the traveling information DB 303 is acquired. Here, the acquired traveling information includes the traveling history of the vehicle 11 and the number of changes in the scheduled traveling route of the vehicle 11 during a period from when the vehicle 11 entered the determination range until the current time. Next, at S104, it is determined whether the traveling situation of the vehicle 11 satisfies a predetermined traveling condition or not (that is, the predetermined determination process is executed) based on the traveling information acquired at S103. If a negative judgment is made at S104, it can be judged that the user does not miss his way at present. In this case, the notification process is ended once. On the other hand, if a positive judgment is made at S104, it can be judged that the user misses his way. Therefore, a process of S105 is executed next.

At S105, the notification information for prompting the operator to perform route guidance for the user by a telephone conversation is transmitted to the operator terminal 200. In the operator terminal 200, when the notification information is received from the management server 300, the notification information is displayed on the display unit 202. Thereby, the operator can grasp that the user who is moving by the vehicle 11 misses his way. Next, at S106, a communication line for a telephone conversation between the user and the operator is connected between the onboard terminal 100 and the operator terminal 200. After that, the notification process is ended.

When judging that the traveling situation of the vehicle 11 satisfies a predetermined traveling condition in the predetermined determination process, the management server 300 in the present embodiment transmits the notification information to the operator terminal 200. Then, by the notification information being displayed on the display unit 202 of the operator terminal 200, it is possible to prompt the operator to perform route guidance for the user who misses his way. Thereby, it becomes possible to, when the user who is moving by the vehicle 11 misses his way, guide the user to the destination by the operator's telephone conversation.

Further, when moving toward the destination by the vehicle 11, the user may intentionally stop in at a place other than the destination at a location relatively away from the destination or cause the vehicle 11 to travel, deviating from the scheduled traveling route to the destination. In such a case, since the user does not miss his way, he does not need route guiding by the operator. Therefore, in the present embodiment, the predetermined determination process is executed when the vehicle 11 exists within the determination range, in the notification process. In other words, when the vehicle 11 exists outside the determination range, the predetermined determination process is not executed. Therefore, for example, even if the vehicle 11 stops for a predetermined time or more when the user stops in at a place other than the destination, which is located outside the determination range, transmission of the notification information from the management server 300 to the operator terminal 200 is not performed. Thus, by the notification prompting route guidance for the user by a telephone conversation is not given to the operator when the vehicle 11 exists outside the determination range, it is possible to prevent an unnecessary telephone conversation for route guidance from being performed for the user.

### Second Embodiment

In the first embodiment, if it is judged in the predetermined determination process that the traveling situation of the vehicle 11 existing in the determination range satisfies a predetermined traveling condition, the management server 300 transmits the notification information to the operator terminal 200. In the present embodiment, before transmitting the notification information to the operator terminal 200, the management server 300 gives a notification confirming whether or not to request route guidance by the operator to the user.

Since a functional configuration of each of the onboard terminal 100, the operator terminal 200 and the management server 300 included in the telephone conversation management system 1 in the present embodiment is similar to the first embodiment, description will be omitted.

(Confirmation to User)

In the present embodiment, if judging that the traveling situation of the vehicle 11 existing in the determination range satisfies a predetermined traveling condition, the control unit 301 in the management server 300 transmits confirmation information for confirming whether or not to request route guidance by the operator to the user, to the onboard terminal 100.

In the onboard terminal 100, the confirmation information is displayed on the display unit that the navigation unit 103 has. Thereby, it becomes possible for the user who misses his way to request route guidance by the operator. Specifically, the user who needs route guidance by the operator inputs information indicating that he requests route guidance, to an input unit that the navigation unit 103 has. Then, when the information indicating that the user requests route guidance is inputted by the user, the control unit 101 transmits request information indicating that route guidance has been requested, to the management server 300 via the communication unit 106. When receiving the request information from the onboard terminal 100, the management server 300 transmits the notification information to the operator terminal 200. On the other hand, if the user does not need route guidance by the operator, the information indicating that the user requests route guidance is not inputted to the navigation unit 103 in the onboard terminal 100. Therefore, the request information is not transmitted from the onboard terminal 100 to the management server 300. In this case, transmission of the notification information from the management server 300 to the operator terminal 200 is not performed, either.

Note that the confirmation information does not necessarily have to be displayed the display unit that the navigation unit 103 has. For example, the user may be notified of the content of the confirmation information by speech output from the speech output unit 105. Further, the information indicating that the user requests route guidance may be inputted to an input device other than the input unit that the navigation unit 103 has. For example, the user may input the information indicating that he requests route guidance by inputting a speech to the speech input unit 104.

(Flow of Notification Process)

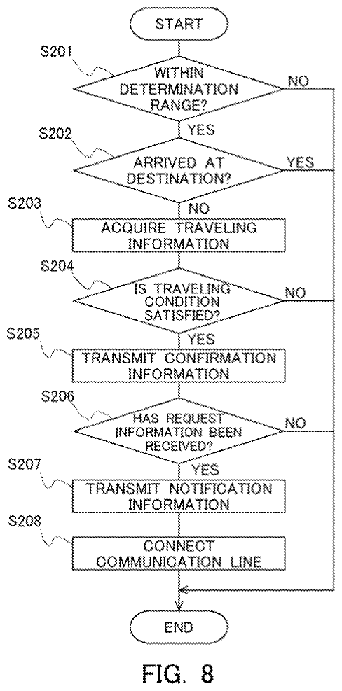

A notification process executed by the control unit 301 in the management server 300, in the present embodiment will be described based on FIG. 8. FIG. 8 is a flowchart illustrating a flow of the notification process in the present embodiment. Note that the process illustrated in FIG. 8 is cyclically executed at predetermined intervals. Note that, since a process from S201 to S204 in the present embodiment is the same as the process from S101 to S104 in the first embodiment, description will be omitted. Further, since processes of S207 and S208 in the present embodiment are also similar to the processes of S105 and S106 in the first embodiment, description will be omitted.

If a negative judgment is made at S204, a process of S205 is executed. At S205, the confirmation information for confirming whether or not to request route guidance by the operator is transmitted to the onboard terminal 100.

Next, at S206, it is determined whether or not the request information has been received from the onboard terminal 100. If a negative judgment is made at S206, the notification process is ended once. On the other hand, if a positive judgment is made at S206, that is, the request information has been received from the onboard terminal 100, a process of S207 is executed. In other words, similarly to the first embodiment, the notification information for prompting the operator to perform route guidance for the user by a telephone conversation is transmitted to the operator terminal 200. After that, the notification process is ended.

In the present embodiment, if judging that the traveling situation of the vehicle 11 satisfies a predetermined traveling condition, the management server 300 transmits the confirmation information to the onboard terminal 100. Then, only when receiving the request information from the onboard terminal 100, the management server 300 transmits the notification information to the operator terminal 200. Thereby, only when the user needs route guidance by a telephone conversation with the operator, the management server 300 can prompt the operator to perform route guidance for the user who misses his way. Therefore, if the user does not need route guidance by the operator, a telephone conversation from the operator is not performed for the user. Therefore, it is possible to prevent an unnecessary telephone conversation by the operator from being performed for the user.

(Modification)

In the first and second embodiments described above, it is determined whether the traveling situation of the vehicle 11 satisfies a predetermined traveling condition or not as the predetermined determination process for determining whether the user misses his way or not. However, a condition by which it can be determined whether the user misses his way or not is not limited to a condition about the traveling situation of the vehicle 11. In the present modification, the management server 300 determines whether a predetermined condition (which may be hereinafter referred to as “biometric condition”) is satisfied or not for biometric information about the user as the predetermined determination process.

(System Configuration)

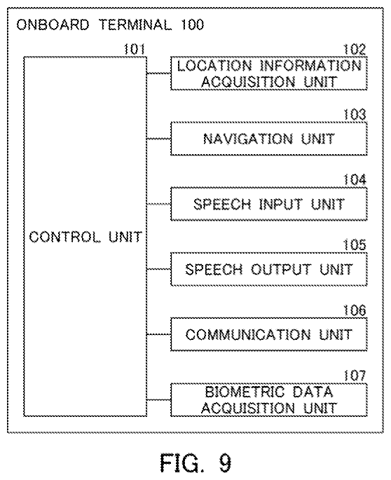

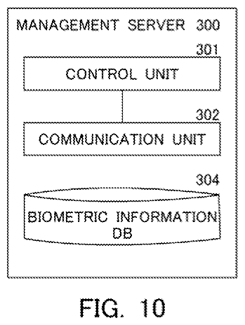

A functional configuration of each of the onboard terminal 100 and the management server 300 included in the telephone conversation management system 1 according to the present modification will be described based on FIGS. 9 and 10. Note that, since a functional configuration of the operator terminal 200 included in the telephone conversation management system 1 is similar to the first embodiment, description will be omitted.

FIG. 9 is a block diagram schematically illustrating an example of the functional configuration of the onboard terminal 100. The onboard terminal 100 is configured, including the control unit 101, the location information acquisition unit 102, the navigation unit 103, the speech input unit 104, the speech output unit 105, the communication unit 106 and a biometric data acquisition unit 107. In the present modification, since functions of the control unit 101, the location information acquisition unit 102, the navigation unit 103, the speech input unit 104 and the speech output unit 105 are similar to the first embodiment, description will be omitted.

The biometric data acquisition unit 107 is a device that acquires biometric data of the user. In the present modification, the biometric data acquisition unit 107 acquires a face image, heart rate, blood pressure and sweat rate of the user as the biometric data of the user. At this time, the biometric data acquisition unit 107 may be provided with a camera that photographs the user's face image, a heart rate monitor, a blood pressure gauge and a sweat rate meter. The control unit 101 transmits the biometric data of the user acquired by the biometric data acquisition unit 107 to the management server 300 via the communication unit 106. Note that the biometric data acquired from the biometric data acquisition unit 107 may be biometric data other than the face image, heart rate, blood pressure and sweat rate of the user.

FIG. 10 is a block diagram schematically illustrating an example of the functional configuration of the management server 300. In the present modification, the management server 300 is configured, including the control unit 301, the communication unit 302 and a biometric information database (a biometric information DB) 304. The control unit 301 receives the biometric data of the user from the onboard terminal 100 via the communication unit 302. Further, similarly to the first embodiment, the control unit 301 transmits the notification information to the operator terminal 200 via the communication unit 302.

After the vehicle 11 enters the determination range, the control unit 301 continuously receives the biometric data of the user from the onboard terminal 100 via the communication unit 302. Then, the control unit 301 calculates a line-of-sight direction from the user's face image among the continuously received biometric data. Then, the control unit 301 generates information about transition of the line-of-sight direction of the user. Further, the control unit 301 generates information about transition of the heart rate of the user, transition of the blood pressure and transition of the sweat rate from information about the heart rate, blood pressure and sweat rate of the user among the continuously received biometric data of the user. In other words, the control unit 301 generates information about transition of the biometric data of the user.

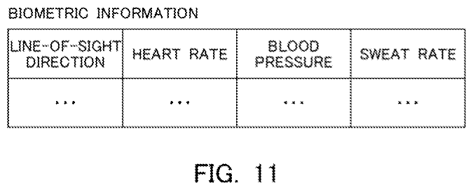

The control unit 301 stores the information about the transition of the biometric data of the user into the biometric information DB 304 as the biometric information. FIG. 11 is a diagram illustrating an example of a table configuration of a biometric information stored in the biometric information DB 304. The biometric information table has a line-of-sight direction field, a heart rate field, a blood pressure field and a sweat rate field. In the line-of-sight direction field, information about transition of the line-of-sight direction of the user is inputted. In the heart rate field, information about transition of the heart rate of the user is inputted. In the blood pressure field, information about transition of the blood pressure of the user is inputted. In the sweat rate field, information about transition of the sweat rate of the user is inputted.

(Predetermined Determination Process)

In the management server 300, the control unit 301 determines whether a predetermined biometric condition is satisfied or not for the biometric information about the user, as the predetermined determination process for determining whether the user misses his way or not. The determination about whether a predetermined biometric condition is satisfied or not for the biometric information about the user is performed based on the biometric information stored in the biometric information DB 304. The predetermined biometric condition is a condition by which it can be judged that the user misses his way. The “predetermined biometric condition” at this time will be specifically described below.

For example, when the user misses his way, he may look around himself to find an intersection, a building, a signboard or the like to be a landmark. In this case, the line-of-sight direction of the user drastically changes. Therefore, the predetermined biometric condition may be that transition of the line-of-sight direction of the user changes more drastically than usual.

Further, when the user misses his way, he may be in a tense state. The control unit 301 can grasp whether the user is in the tense state from transitions of the heart rate, blood pressure and sweat rate of the user. Therefore, the predetermined biometric condition may be that at least any of the transition of the heart rate of the user, the transition of the blood pressure and the transition of the sweat rate changes more drastically than usual.

If at least any one of the predetermined biometric conditions described above is satisfied for the biometric information about the user, it can be judged that the user misses his way. Note that the predetermined biometric conditions described above are examples of the biometric condition for determining whether the user misses his way or not. As the predetermined biometric condition for determining whether the user misses his way or not, other known conditions can be adopted.

(Notification Process)

In the management server 300 according to the present modification, a notification process is executed, with the process for determining whether a predetermined biometric condition is satisfied or not for the biometric information about the user as described above as the predetermined determination process. In other words, instead of S103 and S104 in the flow illustrated in FIG. 7 if the present modification is applied to the first embodiment, and instead of S203 and S204 in the flow illustrated in FIG. 8 if the present modification is applied to the second embodiment, the process for acquiring the biometric information about the user from the biometric information DB 304 and the process for determining whether a predetermined biometric condition is satisfied or not for the acquired biometric information about the user are executed.

Note that the predetermined determination process for determining whether the user misses his way or not may be performed based on both of the traveling information about the vehicle 11 and the biometric information about the user. In this case, it becomes possible to prompt the operator to perform route guidance for the user who misses his way with a higher accuracy.

### Other Embodiments

The embodiments described above are merely examples, and the present disclosure can be appropriately changed within a range not departing from the spirit thereof and implemented. The processes and means described in the present disclosure can be freely combined and implemented unless technical contradiction occurs.

A process described as being performed by one apparatus may be shared and executed by a plurality of apparatuses. Or otherwise, a process described as being performed by different apparatuses may be executed by one apparatus. In a computer system, by what hardware configuration (server configuration) each function is to be realized is flexibly changeable.

The present disclosure can be realized by supplying a computer program in which the functions described in the embodiments and modification described above are implemented to a computer, and one or more processors that the computer has reading out and executing the program. Such a computer program may be provided to the computer by a non-transitory computer-readable storage medium connectable to a system bus of the computer or may be provided to the computer via a network. As the non-transitory computer-readable storage medium, for example, a disk of an arbitrary type such as a magnetic disk (a floppy (R) disk, a hard disk drive (HDD) and the like) and an optical disk (a CD-ROM, a DVD disk, A Blu-ray disk and the like), a read-only memory (ROM), a random access memory (RAM), an EPROM, an EEPROM, a magnetic card, a flash memory, an optical card, and a medium of an arbitrary type that is appropriate for storing electronic commands are included.

### DESCRIPTION OF THE REFERENCE NUMERALS AND SYMBOLS

1: telephone conversation management system
11: vehicle
100: onboard terminal
200: operator terminal
300: management server
301: control unit

What is claimed is:

1. An information processing apparatus managing a communication line for a telephone conversation between a user who is driving a vehicle and an operator who makes a telephone conversation with the user, the information processing apparatus comprising a controller comprising at least one processor configured to:
execute a predetermined determination process for determining whether a predetermined condition by which the user can be judged to miss his way is met when a current location of the vehicle is within a predetermined range from a destination of the user; and
notify the operator to perform route guidance by a telephone conversation for the user if judging that the predetermined condition is met in the predetermined determination process.

2. The information processing apparatus according to claim 1, wherein
if judging that the predetermined condition is met in the predetermined determination process, the controller further executes giving a notification confirming whether or not to request the route guidance by the operator, to the user;
if the route guidance by the operator is requested by the user, the controller notifies the operator to perform the route guidance by the telephone conversation for the user; and
if the route guidance by the operator is not requested by the user, the controller does not notify the operator.

3. The information processing apparatus according to claim 1, wherein the predetermined condition includes a condition about a traveling situation of the vehicle.

4. The information processing apparatus according to claim 3, wherein
the predetermined condition includes at least any one of:
the vehicle stopping at a position different from the destination for a predetermined time or more;
the vehicle turning;
the vehicle entering a one-way road from an opposite direction;
the vehicle traveling away from the destination;
the vehicle traveling on a road or intersection where the vehicle has traveled once, again;
the vehicle not having arrived at the destination even if a predetermined time passes after a scheduled arrival time at which the vehicle is to arrive at the destination, the scheduled arrival time having been estimated when the vehicle enters a predetermined range from the destination; and
a scheduled traveling route estimated as a traveling route of the vehicle from the current location of the vehicle to the destination being changed a predetermined number of times or more.

5. The information processing apparatus according to claim 1, wherein the predetermined condition includes a condition about biometric information about the user.

6. The information processing apparatus according to claim 5, wherein the biometric information includes information about at least any one of a line-of-sight direction of the user, a heart rate of the user, a blood pressure of the user and a sweat rate of the user.

7. The information processing apparatus according to claim 1, wherein the predetermined condition includes conditions about a traveling situation of the vehicle and biometric information about the user.

8. An information processing method for managing a communication line for a telephone conversation between a user who is driving a vehicle and an operator who makes a telephone conversation with the user, the information processing method comprising the steps of:
executing a predetermined determination process for determining whether a predetermined condition by which the user can be judged to miss his way is met when a current location of the vehicle is within a predetermined range from a destination of the user; and
notifying the operator to perform route guidance by a telephone conversation for the user if judging that the predetermined condition is met in the predetermined determination process.

9. The information processing method according to claim 8, further comprising a step of, if judging that the predetermined condition is met in the predetermined determination process, giving a notification confirming whether or not to request the route guidance by the operator, to the user; wherein
if the route guidance by the operator is requested by the user, the operator is notified to perform the route guidance by the telephone conversation for the user; and
if the route guidance by the operator is not requested by the user, the operator is not notified.

10. The information processing method according to claim 8, wherein the predetermined condition includes a condition about a traveling situation of the vehicle.

11. The information processing method according to claim 10, wherein
the predetermined condition includes at least any one of:
the vehicle stopping at a position different from the destination for a predetermined time or more;
the vehicle turning;
the vehicle entering a one-way road from an opposite direction;
the vehicle traveling away from the destination;
the vehicle traveling on a road or intersection where the vehicle has traveled once, again;
the vehicle not having arrived at the destination even if a predetermined time passes after a scheduled arrival time at which the vehicle is to arrive at the destination, the scheduled arrival time having been estimated when the vehicle enters a predetermined range from the destination; and
a scheduled traveling route estimated as a traveling route of the vehicle from the current location of the vehicle to the destination being changed a predetermined number of times or more.

12. The information processing method according to claim 8, wherein the predetermined condition includes a condition about biometric information about the user.

13. The information processing method according to claim 12, wherein the biometric information includes information about at least any one of a line-of-sight direction of the user, a heart rate of the user, a blood pressure of the user and a sweat rate of the user.

14. The information processing method according to claim 8, wherein the predetermined condition includes conditions about a traveling situation of the vehicle and biometric information about the user.

15. A system comprising:
an information processing apparatus configured to manage a communication line for a telephone conversation between a user who is driving a vehicle and an operator who makes a telephone conversation with the user;
an onboard terminal mounted on the vehicle; and
an operator terminal that the operator uses; wherein
the information processing apparatus executes a predetermined determination process for determining whether a predetermined condition by which the user can be judged to miss his way is met when a current location of the vehicle received from the onboard terminal is within a predetermined range from a destination of the user; and
if judging that the predetermined condition is met in the predetermined determination process, the information processing apparatus transmits notification information for prompting the operator to perform route guidance by a telephone conversation for the user, to the operator terminal.

16. The system according to claim 15, wherein
if judging that the predetermined condition is met in the predetermined determination process, the information processing apparatus transmits confirmation information for confirming whether or not to request the route guidance by the operator, to the user, to the onboard terminal; and
if receiving request information indicating that the route guidance by the operator is requested, from the onboard terminal, the information processing apparatus transmits the notification information to the operator terminal; and
if not receiving the request information from the onboard terminal, the information processing apparatus does not transmit the notification information to the operator terminal.

17. The system according to claim 15, wherein
the predetermined condition includes a condition about a traveling situation of the vehicle; and
the information processing apparatus performs determination about whether the traveling situation of the vehicle satisfies the predetermined condition or not based on traveling information about the vehicle received from the onboard terminal in the predetermined determination process.

18. The system according to claim 17, wherein
the predetermined condition includes at least any one of:
the vehicle stopping at a position different from the destination for a predetermined time or more;
the vehicle turning;
the vehicle entering a one-way road from an opposite direction;
the vehicle traveling away from the destination;
the vehicle traveling on a road or intersection where the vehicle has traveled once, again;
the vehicle not having arrived at the destination even if a predetermined time passes after a scheduled arrival time at which the vehicle is to arrive at the destination, the scheduled arrival time having been estimated when the vehicle enters a predetermined range from the destination; and
a scheduled traveling route estimated as a traveling route of the vehicle from the current location of the vehicle to the destination being changed a predetermined number of times or more.

19. The system according to claim 15, wherein
the predetermined condition includes a condition about biometric information about the user; and
the information processing apparatus determines whether the biometric information about the user received from the onboard terminal satisfies the predetermined condition or not in the predetermined determination process.

20. The system according to claim 15, wherein the predetermined condition includes conditions about a traveling situation of the vehicle and biometric information about the user.
